(19) United States
(12) Patent Application Publication
(10) Pub. No.: US 2023/0321066 A1
(43) Pub. Date: Oct. 12, 2023

(54) PREVENTATIVE TREATMENT OF MIGRAINE

(71) Applicant: Biohaven Pharmaceutical Ireland DAC, New Haven, CT (US)

(72) Inventors: Robert CROOP, Newton Square, PA (US); Vladimir CORIC, Madison, CT (US)

(21) Appl. No.: 17/912,545

(22) PCT Filed: Mar. 29, 2021
(86) PCT No.: PCT/US21/24551

§ 371 Date: Sep. 19, 2022

Related U.S. Application Data
(60) Provisional application No. 63/001,341, filed on Mar. 29, 2020.
(60) Provisional application No. 63/111,138, filed on Nov. 9, 2020.
(60) Provisional application No. 63/125,247, filed on Dec. 14, 2020.

(51) Int. Cl. A61K 31/4545; A61K 45/06; A61K 9/20; A61P 25/06

(52) U.S. Cl. CPC A61K 31/4545; A61K 45/06; A61K 9/2054; A61P 25/06

(57) ABSTRACT

Disclosed are methods of for the preventative treatment of migraine, by administering to a patient in need thereof rimegepant or a pharmaceutically acceptable salt thereof. Pharmaceutical compositions comprising rimegepant and kits including the pharmaceutical compositions and instructions are also disclosed.

### CROSS-REFERENCE TO RELATED APPLICATIONS

This application claims priority to U.S. Provisional Application No. 63/001,341 filed Mar. 29, 2020, U.S. Provisional Application No. 63/111,138 filed Nov. 9, 2020, U.S. Provisional Application No. 63/125,247 filed Dec. 14, 2020, and all the benefits accruing therefrom under 35 U.S.C. § 119, the contents of which are incorporated herein in their entireties by reference.

### FIELD OF THE INVENTION

The present invention relates to the use of rimegepant and salts thereof for the preventative treatment of migraine.

### BACKGROUND OF THE INVENTION

Migraine is a chronic and debilitating disorder characterized by recurrent attacks lasting four to 72 hours with multiple symptoms, including typically one-sided, pulsating headaches of moderate to severe pain intensity that are associated with nausea or vomiting, and/or sensitivity to sound (phonophobia) and sensitivity to light (photophobia). Migraines are often preceded by transient neurological warning symptoms, known as auras, which typically involve visual disturbances such as flashing lights, but may also involve numbness or tingling in parts of the body. Migraine is both widespread and disabling. The Migraine Research Foundation ranks migraine as the world's third most prevalent illness, and the Global Burden of Disease Study 2015 rates migraine as the seventh highest specific cause of disability worldwide. According to the Migraine Research Foundation, in the United States, approximately 36 million individuals suffer from migraine attacks. While most sufferers experience migraine attacks once or twice per month, more than 4 million people have chronic migraine, defined as experiencing at least 15 headache days per month, of which at least eight are migraine, for more than three months. Others have episodic migraine, which is characterized by experiencing less than 15 migraine days per month. People with episodic migraine may progress to chronic migraine over time. Migraine attacks can last four hours or up to three days. More than 90% of individuals suffering from migraine attacks are unable to work or function normally during a migraine attack, with many experiencing comorbid conditions such as depression, anxiety and insomnia. Also, those suffering from migraine often have accompanying nausea and have an aversion to consuming food or liquids during an attack.

CGRP (calcitonin gene-related peptide) is a 37 amino acid neuropeptide, which belongs to a family of peptides that includes calcitonin, adrenomedullin and amylin. In humans, two forms of CGRP (α-CGRP and β-CGRP) exist and have similar activities. They vary by three amino acids and exhibit differential distribution. At least two CGRP receptor subtypes may also account for differential activities. The CGRP receptor is located within pain-signaling pathways, intracranial arteries and mast cells and its activation is thought to play a causal role in migraine pathophysiology. For example, research and clinical studies have shown: serum levels of CGRP are elevated during migraine attacks, infusion of intravenous CGRP produces persistent pain in migraine sufferers and non-migraine sufferers, and treatment with anti-migraine drugs normalizes CGRP activity.

Possible CGRP involvement in migraine has been the basis for the development and clinical testing of a number of compounds, including for example, olcegepant (Boehringer Ingelheim, Ridgefield, CT), telcagepant (Merck Sharp & Dohme Corp., Kenilworth, NJ), ubrogepant (Allergan plc, Dublin, Ireland), rimegepant (Biohaven Pharmaceutical Holding Company Ltd., New Haven, CT), galcanezumab (Eli Lilly and Company, Indianapolis, IN), fremanezumab (Teva Pharmaceutical Industries, Petah Tikva, Israel), eptinezumab (Alder Biopharmaceuticals, Inc., Bothell, WA), and erenumab (Amgen Inc., Thousand Oaks, CA). Another compound recently studied for treatment of migraine is lasmiditan (Eli Lilly and Company, Indianapolis, IN). The U.S. Food and Drug Administration (FDA) has approved several CGRP inhibitors for the treatment of migraine including, NURTEC™ ODT (rimegepant), available from Biohaven Pharmaceutical Holding Company Ltd., New Haven, CT, UBRELVY™ (ubrogepant), available from Allergan plc, Dublin, Ireland, EMGALITY™ (galcanezumab-gnlm), available from Eli Lilly and Company, AJOVY™ (fremanezumab-vfrm) injection, available from Teva Pharmaceutical Industries, VYEPTI™ (eptinezumab-jjmr), available from H. Lundbeck A/S, AIMOVIG™ (erenumab-aooe) injection, available from Amgen Inc. The FDA has also approved a high-affinity 5-HT_{1F} receptor agonist, REYVOW™ (lasmitidan), available from Eli Lilly and Company, Indianapolis, IN.

Currently, clinicians use a number of pharmacologic agents for the acute treatment of migraine. A study published by the American Headache Society in 2015 concluded that the medications deemed effective for the acute treatment of migraine fell into the following classes: triptans, ergotamine derivatives, non-steroidal anti-inflammatory drugs (“NSAIDs”), opioids and combination medications. The current standard of care for the acute treatment of migraine is prescription of triptans, which are serotonin 5-HT_{1B/1D} receptor agonists. Triptans have been developed and approved for the acute treatment of migraine over the past two decades. The initial introduction of triptans represented a shift toward drugs more selectively targeting the suspected pathophysiology of migraine. While triptans account for almost 80% of anti-migraine therapies prescribed at office visits by healthcare providers, issues such as an incomplete effect or headache recurrence remain important clinical limitations. In fact, only about 30% of patients from clinical trials are pain free at two hours after taking triptans. In addition, triptans are contraindicated in patients with cardiovascular disease, cerebrovascular disease, or significant risk factors for either because of potential systemic and cerebrovascular vasoconstriction from the 5-HT_{1B}-mediated effects. Also, according to a January 2017 study published in the journal Headache, an estimated 2.6 million migraine sufferers in the United States have a cardiovascular event, condition or procedure that limits the potential of triptans as a treatment option. In addition, many of the medications prescribed for the treatment of migraine are inadequate for the preventative treatment of migraine.

Accordingly, there remains a significant unmet medical need for the treatment of migraine. In particular, new methods for the preventative treatment of migraine are desired in addition to methods for the acute treatment of migraine.

Patients that suffer from migraine may also suffer from sinusitis. Sinusitis, also known as rhinosinusitis, is inflammation of the mucous membranes that line the sinuses resulting in symptoms. Common symptoms include thick nasal mucus, a plugged nose, and facial pain. Other signs and symptoms may include fever, headaches, a poor sense of smell, sore throat, and a cough. Acute sinusitis typically lasts fewer than 4 weeks, and chronic sinusitis typically lasts for more than 12 weeks. Sinusitis can be caused by viral or bacterial infections, allergies, air pollution, or structural problems in the nose. Sinusitis is believed to affect more than 15% of the U.S. population annually, resulting in over $5.8 billion in direct health care expenditures. Chronic Rhinosinusitis (CRS), represents a large portion of sinusitis cases, affecting more than 30 million Americans. Accordingly, treatments for sinusitis are desired.

### SUMMARY OF THE INVENTION

The present invention is directed, among other things, to the preventative treatment of migraine with rimegepant and salts thereof. By virtue of the present invention, it may now be possible to provide preventative treatment of migraine to patients in need thereof. Quite surprisingly, in accordance with the present invention, patients who are taking a CGRP antagonist for the acute treatment of migraine may experience benefits in migraine prevention when taking rimegepant in accordance with the present invention. Indeed, by taking rimegepant for both acute migraine and migraine prevention, patients may experience synergy in their treatment and more effective relief overall. In accordance with the present invention, rimegepant may have the potential to change the paradigm of migraine treatment, offering patients the potential for a dual acting—acute and prevention—therapy in one simple dose and convenient formulation. For prevention therapy, the ability to take an oral medication every other day rather than a monthly injection or intravenous therapy may provide patients with a less invasive method of treating migraine and may allow patients to feel more in control of their migraine.

In one aspect of the invention, there is provided a method for the preventative treatment migraine in a patient in need thereof, comprising administering to the patient a pharmaceutical composition comprising a pharmaceutically acceptable carrier and a therapeutically effective amount of rimegepant, or a pharmaceutically acceptable salt thereof, at least once every other day in order to provide a reduction in the mean number of migraine days per month for said patient.

In an aspect of the invention, the reduction in the mean number of migraine days per month for said patient at least 20%. In an aspect of the invention, the reduction in the mean number of migraine days per month for said patient at least 30%. In an aspect of the invention, the reduction in the mean number of migraine days per month for said patient at least 40%. In an aspect of the invention, the reduction in the mean number of migraine days per month for said patient at least 50%.

In an aspect of the invention, the pharmaceutical composition is administered once every other day. In an aspect of the invention, the pharmaceutical composition is administered not more than once per day.

In an aspect of the invention, the patient is also administered a medication for the treatment of acute migraine. In an aspect of the invention, the medication is a CGRP inhibitor selected from a CGRP antibody, a CGRP receptor antibody, an antigen-binding fragment from a CGRP antibody or a CGRP receptor antibody, a CGRP infusion inhibitory protein, a CGRP bio-neutralizing agent, a CGRP receptor antagonist, a small molecule CGRP inhibitor, or a polypeptide CGRP inhibitor. In an aspect of the invention, the CGRP antibody is selected from galcanezumab-gnlm, fremanezumab-vfrm, eptinezumab-jjmr, and erenumab-aooe. In an aspect of the invention, the CGRP receptor antagonist is selected from olcegepant, telcagepant, ubrogepant, atogepant, rimegepant, and vazegepant.

In an aspect of the invention, the rimegepant is in the form of a hemisulfate sesquihydrate salt. In an aspect of the invention, the pharmaceutical composition in the form of a tablet. In an aspect of the invention, the pharmaceutical composition comprises from about 50-60 percent by weight (weight %) rimegepant hemisulfate sesquihydrate, about 30-35 weight % microcrystalline cellulose, about 2-7 weight % hydroxypropyl cellulose, about 3-7 weight % croscarmellose sodium, and about 0.1-1.0 weight % magnesium stearate. In an aspect of the invention, the pharmaceutical composition comprises about 57.1 weight % rimegepant hemisulfate sesquihydrate, about 33.4 weight % microcrystalline cellulose, about 4.0 weight % hydroxypropyl cellulose, about 5.0 weight % croscarmellose sodium, and about 0.5 weight % magnesium stearate. In an aspect of the invention, the pharmaceutical composition is in the form of an oral solid molded fast-dispersing dosage form. In an aspect of the invention, the pharmaceutical composition comprises from about 70-80 weight % rimegepant hemisulfate sesquihydrate, about 10-20 weight % fish gelatin, about 10-20 weight % of a filler, and 0.1-5.0 weight % of a flavorant. In an aspect of the invention, the filler is mannitol.

In an aspect of the invention, there is provided a method of treating sinusitis in a patient in need thereof, comprising administering to the patient a pharmaceutical composition comprising a pharmaceutically acceptable carrier and a therapeutically effective amount of rimegepant, or a pharmaceutically acceptable salt thereof, in order to relieve a symptom associated with the sinusitis. In an aspect of the invention, the treatment is effective to provide pain relief to the patient. In an aspect of the invention, the treatment is effective to reduce an infection in the patient. In an aspect of the invention, the treatment is effective to reduce inflammation in the patient.

### DETAILED DESCRIPTION OF THE INVENTION

The following detailed description is provided to aid those skilled in the art in practicing the present invention. Those of ordinary skill in the art may make modifications and variations in the embodiments described herein without departing from the spirit or scope of the present disclosure. Unless otherwise defined, all technical and scientific terms used herein have the same meaning as commonly understood by one of ordinary skill in the art to which this disclosure belongs. The terminology used in the description is for describing particular embodiments only and is not intended to be limiting.

As used in this application, except as otherwise expressly provided herein, each of the following terms shall have the meaning set forth below. Additional definitions are set forth throughout the application. In instances where a term is not specifically defined herein, that term is given an art-recognized meaning by those of ordinary skill applying that term in context to its use in describing the present invention.

The articles “a” and “an” refer to one or to more than one (i.e., to at least one) of the grammatical object of the article unless the context clearly indicates otherwise. By way of example, “an element” means one element or more than one element.

The term “about” refers to a value or composition that is within an acceptable error range for the particular value or composition as determined by one of ordinary skill in the art, which will depend in part on how the value or composition is measured or determined, i.e., the limitations of the measurement system. For example, “about” can mean within 1 or more than 1 standard deviation per the practice in the art. Alternatively, “about” can mean a range of up to 1%, 5%, 10% or 20% (i.e., ±10% or ±20%) depending on the context of the application. For example, about 3 mg can include any number between 2.7 mg and 3.3 mg (for 10%) or between 2.4 mg and 3.6 mg (for 20%). Furthermore, particularly with respect to biological systems or processes, the terms can mean up to an order of magnitude or up to 5-fold of a value. When particular values or compositions are provided in the application and claims, unless otherwise stated, the meaning of “about” should be assumed to be within an acceptable error range for that particular value or composition.

The term “administering” refers to the physical introduction of a composition comprising a therapeutic agent to a subject, using any of the various methods and delivery systems known to those skilled in the art. Administering can also be performed, for example, once, a plurality of times, and/or over one or more extended periods and can be a therapeutically effective dose or a subtherapeutic dose.

The term “antibody” (Ab) refers to, without limitation, a glycoprotein immunoglobulin which binds specifically to an antigen and comprises at least two heavy (H) chains and two light (L) chains interconnected by disulfide bonds, or an antigen-binding portion thereof. Each H chain comprises a heavy chain variable region (abbreviated herein as V_{H}) and a heavy chain constant region. The heavy chain constant region comprises three constant domains, C_{H1}, C_{H2} and C_{H3}. Each light chain comprises a light chain variable region (abbreviated herein as V_{L}) and a light chain constant region. The light chain constant region comprises one constant domain, C_{L}. The V_{H} and V_{L} regions can be further subdivided into regions of hypervariability, termed complementarity determining regions (CDRs), interspersed with regions that are more conserved, termed framework regions (FR). Each V_{H} and V_{L} comprises three CDRs and four FRs, arranged from amino-terminus to carboxy-terminus in the following order: FR1, CDR1, FR2, CDR2, FR3, CDR3, FR4. The variable regions of the heavy and light chains contain a binding domain that interacts with an antigen. The constant regions of the antibodies can mediate the binding of the immunoglobulin to host tissues or factors, including various cells of the immune system (e.g., effector cells) and the first component (C1q) of the classical complement system.

An immunoglobulin can derive from any of the commonly known isotypes, including but not limited to IgA, secretory IgA, IgG and IgM. IgG subclasses are also well known to those in the art and include but are not limited to human IgG1, IgG2, IgG3 and IgG4. As used herein, the term “isotype” refers, without limitation, to the antibody class or subclass (e.g., IgM or IgG1) that is encoded by the heavy chain constant region genes. In certain embodiments, one or more amino acids of the isotype can be mutated to alter effector function. As used herein, the term “antibody” includes, by way of example, both naturally occurring and non-naturally occurring Abs; monoclonal and polyclonal Abs; chimeric and humanized Abs; human or nonhuman Abs; wholly synthetic Abs; and single chain antibodies. A nonhuman antibody can be humanized by recombinant methods to reduce its immunogenicity in man. Where not expressly stated, and unless the context indicates otherwise, the term “antibody” also includes an antigen-binding fragment or an antigen-binding portion of any of the aforementioned immunoglobulins, and includes a monovalent and a divalent fragment or portion, and a single chain antibody.

The terms “in combination with” and “in conjunction with” refer to administration of one treatment modality in addition to another treatment modality. As such, “in combination with” or “in conjunction with” refers to administration of one treatment modality before, during, or after administration of the other treatment modality to the subject.

The term “pharmaceutically acceptable salt” refers to a salt form of one or more of the compounds described herein which are typically presented to increase the solubility of the compound in the gastric or gastroenteric juices of the patient's gastrointestinal tract in order to promote dissolution and the bioavailability of the compounds. Pharmaceutically acceptable salts include those derived from pharmaceutically acceptable inorganic or organic bases and acids, where applicable. Suitable salts include, for example, those derived from alkali metals such as potassium and sodium, alkaline earth metals such as calcium, magnesium and ammonium salts, among numerous other acids and bases well known in the pharmaceutical art.

The terms “subject” and “patient” refer any human or nonhuman animal. The term “nonhuman animal” includes, but is not limited to, vertebrates such as nonhuman primates, sheep, dogs, and rodents such as mice, rats and guinea pigs. In some embodiments, the subject is a human. The terms, “subject” and “patient” are used interchangeably herein.

The terms “effective amount”, “therapeutically effective amount”, “therapeutically effective dosage” and “therapeutically effective dose” of an agent (also sometimes referred to herein as a “drug”) refers to any amount of the agent that, when used alone or in combination with another agent, protects a subject against the onset of a disease or promotes disease regression evidenced by a decrease in severity of disease symptoms, an increase in frequency and duration of disease symptom-free periods, or relief from impairment or disability due to the disease affliction. The therapeutically effective amount of an agent can be evaluated using a variety of methods known to the skilled practitioner, such as in human subjects during clinical trials, in animal model systems predictive of efficacy in humans, or by assaying the activity of the agent in in vitro assays.

The term “treatment” refers to any treatment of a condition or disease in a subject and may include: (i) preventing the disease or condition from occurring in the subject which may be predisposed to the disease but has not yet been diagnosed as having it; (ii) inhibiting the disease or condition, i.e., arresting its development; relieving the disease or condition, i.e., causing regression of the condition; or (iii) ameliorating or relieving the conditions caused by the disease, i.e., symptoms of the disease. Treatment could be used in combination with other standard therapies or alone. Treatment or “therapy” of a subject also includes any type of intervention or process performed on, or the administration of an agent to, the subject with the objective of reversing, alleviating, ameliorating, inhibiting, slowing down or preventing the onset, progression, development, severity or recurrence of a symptom, complication or condition, or biochemical indicia associated with a disease.

With respect to headache, “treatment” is an approach for obtaining beneficial or desired results with a subject. For purposes of this invention, beneficial or desired clinical results include, but are not limited to, one or more of the following: improvement in any aspect of a headache including lessening severity, alleviation of pain intensity, and other associated symptoms, reducing frequency of recurrence, increasing the quality of life of those suffering from the headache, decreasing dose of other medications required to treat the headache and reducing the number of headache days per month. For migraine, other associated symptoms include, but are not limited to, nausea, vomiting, and sensitivity to light, sound, and/or movement. For cluster headache, other associated symptoms include, but are not limited to swelling under or around the eyes, excessive tears, red eye, Rhinorrhea or nasal congestion, and red flushed face.

For purposes of this disclosure, reference is made to the publication by the U.S. Food and Drug Administration (FDA), Guidance for Industry, “Migraine: Developing Drugs for Acute Treatment”, February 2018, available from https://www.fda.gov/downloads/drugs/guidances/ucm419465.pdf. Terms used in the Examples, such as, for example, most bothersome symptoms (MBS) and Pain Freedom, are described in the FDA Guidance.

The starting materials useful for making the pharmaceutical compositions of the present invention are readily commercially available or can be prepared by those skilled in the art.

Rimegepant has the chemical formula, C₂₈H₂₈F₂N₆O₃ and the IUPAC name [(5S,6S,9R)-5-amino-6-(2,3-difluorophenyl)-6,7,8,9-tetrahydro-5H-cyclohepta[b]pyridin-9-yl] 4-(2-oxo-3H-imidazo[4,5-b]pyridin-1-yl)piperidine-1-carboxylate. Rimegepant is also referred to herein as BHV-3000.

The structure of rimegepant is:

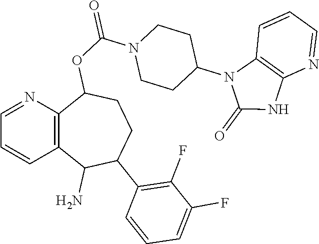 [SMILES: NC1c2cccnc2C(OC(=O)N2CCC(n3c(=O)[nH]c4ncccc43)CC2)CCC1c1cccc(F)c1F]

Rimegepant is described, for example in WO 2011/046997 published April 21, 2011.

In a preferred aspect of the invention, rimegepant is present in the form of a hemisulfate sesquihydrate salt. This preferred salt form is described in WO 2013/130402 published Sep. 6, 2013.

The chemical formula of the salt form is C₂₈H₂₈F₂N₆O₃·0.5 H₂SO₄·1.5 H₂O and the structure is as follows:

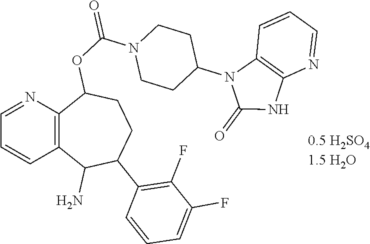 [SMILES: NC1c2cccnc2C(OC(=O)N2CCC(n3c(=O)[nH]c4ncccc43)CC2)CCC1c1cccc(F)c1F]

Rimegepant is available under the brand name NURTEC™ ODT (rimegepant) from Biohaven Pharmaceutical Holding Company Ltd., New Haven, CT.

The pharmaceutical compositions of the present invention can be prepared in any suitable dosage form including, for example, such as tablets, capsules, nasal sprays, powders, granules, ointments, solutions, suppositories, injections, inhalants, gels, microspheres, and aerosols.

The pharmaceutical compositions of the present invention comprising rimegepant typically also include other pharmaceutically acceptable carriers (also referred to as excipients) such as, for example, binders, lubricants, diluents, coatings, disintegrants, barrier layer components, glidants, coloring agents, solubility enhancers, gelling agents, fillers, proteins, co-factors, emulsifiers, solubilizing agents, suspending agents, flavorants, preservatives and mixtures thereof. The choice of excipients depends on the desired characteristics of the compositions and on the nature of other pharmacologically active compounds in the formulation. Suitable excipients are known to those skilled in the art (see Handbook of Pharmaceutical Excipients, fifth edition, 2005 edited by Rowe et al., McGraw Hill).

Examples of pharmaceutically acceptable carriers that may be used in preparing the pharmaceutical compositions of the present invention may include, but are not limited to, fillers such as sugars, including lactose, sucrose, mannitol, or sorbitol; cellulose preparations such as maize starch, wheat starch, rice starch, potato starch, gelatin, gum tragacanth, methyl cellulose, hydroxypropyl methyl-cellulose, sodium carboxymethylcellulose, polyvinyl-pyrrolidone (PVP), talc, calcium sulphate, vegetable oils, synthetic oils, polyols, alginic acid, phosphate buffered solutions, emulsifiers, isotonic saline, pyrogen-free water and combinations thereof. If desired, disintegrating agents may be combined as well, and exemplary disintegrating agents may be, but not limited to, cross-linked polyvinyl pyrrolidone, agar, or alginic acid or a salt thereof such as sodium alginate. In an aspect of the invention, the flavoring agent is selected from mint, peppermint, berries, cherries, menthol and sodium chloride flavoring agents, and combinations thereof. In an aspect of the invention, the sweetener is selected from sugar, sucralose, aspartame, acesulfame, neotame, and combinations thereof.

In general, the pharmaceutical compositions of the present invention may be manufactured in conventional methods known in the art, for example, by means of conventional mixing, dissolving, granulating, dragee-making, levigating, emulsifying, encapsulating, entrapping, lyophilizing processes and the like.

In one aspect of the invention the pharmaceutical compositions are prepared in oral solid molded fast-dispersing dosage form, such as described in U.S. Pat. No. 9,192,580, issued Nov. 24, 2015.

The phrase “fast-dispersing dosage form” refers to compositions which disintegrate or disperse within 1 to 60 seconds, preferably 1 to 30 seconds, more preferably 1 to 10 seconds and particularly 2 to 8 seconds, after being placed in contact with a fluid. The fluid is preferably that found in the oral cavity, i.e., saliva, as with oral administration.

In a preferred embodiment, the compositions of the invention are solid fast-dispersing dosage forms comprising a solid network of the active ingredient, rimegepant, and a water-soluble or water-dispersible carrier containing fish gelatin. Accordingly, the carrier is inert towards the active ingredient. The network is obtained by subliming solvent from a composition in the solid state, the composition comprising the active ingredient and a solution of the carrier in the solvent. The dosage forms according to the invention can be prepared according to the process disclosed in Gregory et al., U.K. Patent No. 1,548,022 using fish gelatin as the carrier. Accordingly, an initial composition (or admixture) comprising the active ingredient and a solution of the fish gelatin carrier in a solvent is prepared followed by sublimation. The sublimation is preferably carried out by freeze drying the composition. The composition can be contained in a mold during the freeze-drying process to produce a solid form in any desired shape. The mold can be cooled using liquid nitrogen or solid carbon dioxide in a preliminary step prior to the deposition of the composition therein. After freezing the mold and composition, they are next subjected to reduced pressure and, if desired, controlled application of heat to aid in sublimation of solvent. The reduced pressure applied in the process can be below about 4 mm Hg, preferably below about 0.3 mm Hg. The freeze dried compositions can then be removed from the mold if desired or stored therein until later use.

When the process is used with active ingredients and fish gelatin as the carrier, a solid fast-dispersing dosage form is produced having the advantages associated with the use of fish gelatin described herein. Generally, fish gelatin is categorized as being from cold water and warm water fish sources and as being of the gelling or non-gelling variety. The non-gelling variety of fish gelatin, in comparison to gelling fish gelatin and bovine gelatin, contains lower proline and hydroxyproline amino acid content, which are known to be associated with cross-linking properties and gelling ability. Non-gelling fish gelatin can remain at solution concentrations of up to about 40% as well as in temperatures as low as 20° C. In one aspect of the invention, the fish gelatin used in accordance with the invention is preferably obtained from cold water fish sources and is the non-gelling type of fish gelatin. More preferably, in one aspect of the invention, the non-hydrolyzed form of non-gelling fish gelatin is used. In an alternative embodiment, spray-dried non-hydrolyzed non-gelling fish gelatin can be used. Fish gelatins suitable for use in the invention are commercially available.

The compositions according to the invention can also contain, in addition to the active ingredient and fish gelatin carrier, other matrix forming agents and secondary components. Matrix forming agents suitable for use in the present invention include materials derived from animal or vegetable proteins, such as other gelatins, dextrins and soy, wheat and psyllium seed proteins; gums such as acacia, guar, agar, and xanthan; polysaccharides; alginates; carboxymethylcelluloses; carrageenans; dextrans; pectins; synthetic polymers such as polyvinylpyrrolidone; and polypeptide/protein or polysaccharide complexes such as gelatin-acacia complexes.

Other materials which may also be incorporated into the fast-dissolving compositions of the present invention include sugars such as mannitol, dextrose, lactose, galactose, and trehalose; cyclic sugars such as cyclodextrin; inorganic salts such as sodium phosphate, sodium chloride and aluminum silicates; and amino acids having from 2 to 12 carbon atoms such as glycine, L-alanine, L-aspartic acid, L-glutamic acid, L-hydroxyproline, L-isoleucine, L-leucine and L-phenylalanine. One or more matrix forming agents may be incorporated into the solution or suspension prior to solidification (freezing). The matrix forming agent may be present in addition to a surfactant or to the exclusion of a surfactant. In addition to forming the matrix, the matrix forming agent may aid in maintaining the dispersion of any active ingredient within the solution of suspension. This is especially helpful in the case of active agents that are not sufficiently soluble in water and must, therefore, be suspended rather than dissolved. Secondary components such as preservatives, antioxidants, surfactants, viscosity enhancers, coloring agents, flavoring agents, pH modifiers, sweeteners or taste-masking agents may also be incorporated into the fast-dissolving compositions. Suitable coloring agents include red, black and yellow iron oxides and FD & C dyes such as FD&C Blue No. 2 and FD&C Red No. 40 available from Ellis & Everard. Suitable flavoring agents include mint, raspberry, licorice, orange, lemon, grapefruit, caramel, vanilla, cherry and grape flavors and combinations of these. Suitable pH modifiers include the edible acids and bases, such as citric acid, tartaric acid, phosphoric acid, hydrochloric acid, maleic acid and sodium hydroxide. Suitable sweeteners include, for example, sucralose, aspartame, acesulfame K and thaumatin. Suitable taste-masking agents include, for example, sodium bicarbonate, ion exchange resins, cyclodextrin inclusion compounds, adsorbates or microencapsulated actives.

Typical routes of administering the pharmaceutical compositions of the invention include, without limitation, oral, topical, transdermal, inhalation, parenteral, sublingual, buccal, rectal, vaginal, and intranasal. The term parenteral as used herein includes subcutaneous injections, intravenous, intramuscular, intrasternal injection or infusion techniques. Pharmaceutical compositions according to certain embodiments of the present invention are formulated so as to allow the active ingredients contained therein to be bioavailable upon administration of the composition to a patient. Compositions that will be administered to a subject or patient may take the form of one or more dosage units. Actual methods of preparing such dosage forms are known, or will be apparent, to those skilled in this art; for example, see Remington: The Science and Practice of Pharmacy, 20th Edition (Philadelphia College of Pharmacy and Science, 2000).

Solid compositions are normally formulated in dosage units providing from about 1 to about 1000 mg of the active ingredient per dose. Some examples of solid dosage units are 0.1 mg, 1 mg, 10 mg, 37.5 mg, 75 mg, 100 mg, 150 mg, 300 mg, 500 mg, 600 mg and 1000 mg. Typical dose ranges in accordance with the present invention include from about 10-600 mg, 25-300 mg, 25-150 mg, 50-100 mg, 60-90 mg, and 70-80 mg. Liquid compositions are generally in a unit dosage range of 1-100 mg/mL. Some examples of liquid dosage units are 0.1 mg/mL, 1 mg/mL, 10 mg/mL, 25 mg/mL, 50 mg/mL, and 100 mg/mL.

In some aspects, the invention provides methods of administering to a subject one or more additional agent(s) simultaneously or sequentially with the rimegepant. In some aspects, an additional agent may be an anti-headache medication such as, for example, an anti-headache medication (e.g., 5-HT1 agonists, triptans, ergot alkaloids, opiates, adrenergic antagonists, NSAIDs or antibodies) known in the art.

In some aspects, the additional agent may be topiramate or amitriptyline.

In some aspects, the additional agent may be a biologic, such as those selected from antibodies, antibody fragments or peptides. Such biologics comprise molecules that have a mass typically greater than about 900 Daltons, for example, greater than 1,100 Daltons, greater than 1,300 Daltons, greater than 1,500 Daltons, greater than 5,000 Daltons, greater than 10,000 Daltons, greater than 50,000

Daltons, or greater than 100,000 Daltons. Examples of CGRP biologics commercially available or currently being studied include the following. EMGALITY™ (galcanezumab-gnlm), available from Eli Lilly and Company, is a humanized IgG4 monoclonal antibody specific for calcitonin-gene related peptide (CGRP) ligand. Galcanezumab-gnlm is produced in Chinese Hamster Ovary (CHO) cells by recombinant DNA technology. Galcanezumab-gnlm is composed of two identical immunoglobulin kappa light chains and two identical immunoglobulin gamma heavy chains and has an overall molecular weight of approximately 147 kDa. AJOVY™ (fremanezumab-vfrm) injection, available from Teva Pharmaceutical Industries, is a fully humanized IgG2Da/kappa monoclonal antibody specific for calcitonin gene-related peptide (CGRP) ligand. Fremanezumab-vfrm is produced by recombinant DNA technology in Chinese hamster ovary (CHO) cells. The antibody consists of 1324 amino acids and has a molecular weight of approximately 148 kDa. VYEPTI™ (eptinezumab-jjmr), available from H. Lundbeck A/S, is a fully humanized IgG1 antibody manufactured using yeast (Pichia pastoris). AIMOVIG™ (erenumab-aooe) injection, available from Amgen Inc., is a human immunoglobulin G2 (IgG2) monoclonal antibody that has high affinity binding to the calcitonin gene-related peptide receptor. Erenumab-aooe is produced using recombinant DNA technology in Chinese hamster ovary (CHO) cells. It is composed of 2 heavy chains, each containing 456 amino acids, and 2 light chains of the lambda subclass, each containing 216 amino acids, with an approximate molecular weight of 150 kDa.

In some aspects, the additional agent may be a small molecule CGRP inhibitor. For example, the CGRP inhibitor may be a CGRP receptor antagonist, such as those selected from olcegepant, telcagepant, ubrogepant, atogepant, rimegepant, and vazegepant. Examples of CGRP small molecules commercially available include NURTEC™ ODT (rimegepant), available from Biohaven Pharmaceutical Holding Company Ltd., New Haven, CT, and UBRELVY™ (ubrogepant), available from Allergan plc, Dublin, Ireland. A high-affinity 5-HT_{1F} receptor agonist, REYVOW™ (lasmitidan), is available from Eli Lilly and Company, Indianapolis, IN.

In some aspects, a therapeutic effect may be greater as compared to use of rimegepant or one or more additional agent(s) alone. Accordingly, a synergistic effect between rimegepant and the one or more additional agents may be achieved, for example, in the acute treatment of migraine, the preventative treatment of migraine, or both.

In one aspect, the invention also provides kits for use in the instant methods. Kits can include one or more containers comprising a pharmaceutical composition described herein and instructions for use in accordance with any of the methods described herein. Generally, these instructions comprise a description of administration of the pharmaceutical composition to treat, ameliorate or prevent migraine according to any of the methods described herein. The kit may, for example, comprise a description of selecting an individual suitable for treatment based on identifying whether that individual has headache or whether the individual is at risk of having headache. The instructions are typically provided in the form of a package insert, or label, in accordance with the requirements of the regulatory having authority over the jurisdiction where the pharmaceutical composition is to be provided to patients. For example, the package insert for NURTEC ODT (rimegepant) is available at https://www.nurtec.com/pi.

### EXAMPLES

The following examples illustrate the invention and are not intended to limit the scope of the invention.

### Example 1

Tablet Manufacture—A batch is prepared to manufacture tablets containing a dose of 75 mg of rimegepant as follows. The composition of the batch is set forth below in Table 1. Tablets are made from the batch as indicated.

TABLE 1

| | Percent | Amount | Amount per |
|---|---|---|---|
| | per | per Tablet, | 100,000 Tablet |
| Ingredient | Tablet | (mg) | Batch (g) |
| Intra-granular | | | |
| Rimegepant (as hemisulfate | 57.11 | 85.67 | 8575.5 |
| sesquihydrate equivalent to 75 mg | | | |
| as base) | | | |
| Microcrystalline cellulose, NF | 13.39 | 20.09 | 2,011.0 |
| Hydroxypropyl Cellulose), | 4.00 | 6.00 | 600.6 |
| USP/NF (Klucel EXF PHARM) | | | |
| Croscarmellose Sodium NF | 2.50 | 3.75 | 375.4 |
| Purified Water USP | q.s. | N/A | 0¹ |
| Intragranular Dispensed Solids | | | 11562 |
| Extra-granular | | | |
| Microcrystalline cellulose NF | 20.00 | 30.00 | 3,003.0 |
| Croscarmellose Sodium NF | 2.50 | 3.75 | 375.4 |
| Magnesium Stearate NF | 0.50 | 0.75 | 75.08 |
| Total Core Tablet | 100.0 | 150 | 15015 |

¹Purified Water is removed in-process. An excess amount is dispensed. The portion consumed is documented. Intragranular Dispensed Solids does not include water.

1. The rimegepant hemisulfate sesquihydrate and all excipients are weighed.
2. Pass the rimegepant hemisulfate sesquihydrate, microcrystalline cellulose (intragranular portion), hydroxypropyl cellulose, and croscarmellose sodium (intragranular portion) through a 20-mesh screen.
3. Load the sieved mixture from 2 into a suitable granulator equipped with an appropriate size bowl & dry mix for 10 minutes. Set impeller speed to low & turn chopper off.
4. While mixing, equip the granulator with a spray tip and add purified water until endpoint is reached.
5. Mix wet mass for 30 seconds with impeller set to low and chopper set to low.
6. Discharge the wet mass into expansion chamber of fluid bed dryer. Dry to target LOD of <2%.
7. Mill the dried granules using the Comil with an appropriate screen (0.075R) and spacer (0.050). Perform bulk and tapped density & particle size distribution analyses. Record results. Calculate Carr Index & Carr Index mean from two samples.
8. Calculate the fractional yield. Recalculate the extragranular quantities.
9. Pass the microcrystalline cellulose and croscarmellose through a 20-mesh screen.
10. Combine the milled granulation with the recalculated microcrystalline cellulose (extragranular portion), croscarmellose sodium (extragranular portion) in a 2-cubic foot tote & blend for 150 revolutions.
11. Pass the magnesium stearate through 30-mesh screen.
12. Add screened magnesium stearate to the 2 cubic foot tote contents & blend for 75 revolutions.
13. Collect blend uniformity samples per plan.
14. Perform bulk & tapped density and particle size analyses & calculate Carr Index.
15. Discharge into a suitable container and weigh.
16. Set up 716-station rotary tablet press with 7mm round concave plain tooling. Adjust number of stations as needed.
17. Adjust the press to achieve the following specifications for the tablets: Friability of 0.3% loss; Hardness of 10-14 kP; Thickness of 3.60-4.10 mm; and Disintegration of 2:30 minutes.
18. Conduct in-process testing as follows:
   Tablet friability and disintegration at beginning, middle and end of run
   Tablet hardness, tablet thickness, individual tablet weights, average tablet weights, and appearance at 15 minute intervals
19. Pass the tablets through a de-duster and metal detector.
20. Package tablets in double polyethylene bags in a suitable container.

### Example 2

Clinical Trial—BHV3000-305: A Phase 2/3, Randomized, Double-blind, Placebo-controlled Study to Evaluate the Efficacy and Safety of Rimegepant in Migraine Prevention (ClinicalTrials.gov Identifier: NCT03732638).

A phase 2/3 clinical study was conducted with 1629 participants, as follows.

### Study Description

The purpose of this is study is to compare the efficacy of BHV-3000 (rimegepant) to placebo as a preventive treatment for migraine, as measured by the reduction in the number of migraine days per month.

| Condition or disease | Intervention/treatment | Phase |
|---|---|---|
| Migraine | Drug: Rimegepant/ | Phase 2/Phase 3 |
| | Drug: Placebo | |

### Study Design

Study Type: Interventional (Clinical Trial)
Actual Enrollment: 1629 participants
Allocation: Randomized
Intervention Model: Parallel Assignment
Masking: Triple (Participant, Care Provider, Investigator)
Primary Purpose: Prevention
Official Title: A Phase 2/3, Randomized, Double-blind, Placebo-controlled Study to Evaluate the Efficacy and Safety of Rimegepant in Migraine Prevention
Actual Study Start Date: Nov. 14, 2018
Actual Primary Completion Date: Dec. 10, 2019

Estimated Study Completion Date: Jan. 30, 2021

### Arms and Interventions

| Arm | Intervention/treatment |
|---|---|
| Placebo Comparator:Placebo Comparator | Drug:Placebo |
| | 75 mg matching placebo |

Primary Outcome Measures:
1. Change from baseline in mean number of migraine days per month [Time Frame: Mean number of migraine days per month as assessed over 3 months (12 weeks)]
Change from baseline in mean number of migraine days per month as measured over the 12-week double-blind phase of the study.

Secondary Outcome Measures:
1. Achievement of at least a 50% reduction from baseline in mean number of moderate to severe monthly migraine days. [Time Frame: baseline to end of Week 12]
Achievement of at least a 50% reduction from baseline in mean number of moderate to severe monthly migraine days over course of the double-blind treatment phase.
2. The mean number of rescue medication days per month. [Time Frame: baseline to end of week 12 as measure per month]
The mean number of rescue medication days per month over the course of the double-blind, treatment phase. Rescue medications include the rescue medications defined in this protocol.
3. Adverse events. [Time Frame: baseline to end of Week 12]
Adverse events that occur in at least 5% of treated subjects; the frequency of unique subjects with serious adverse events, adverse events leading to discontinuation and clinically significant laboratory abnormalities, as reported on case report forms.
4. The frequency of AST or ALT elevations. [Time Frame: baseline to end of Week 12]
The frequency of AST or ALT elevations>3×ULN, concurrently with bilirubin elevations>2×ULN, will be assessed by tabulating the number of unique subjects with this pairing of events.
5. The frequency of hepatic-related adverse events and hepatic-related treatment. [Time Frame: baseline to end of Week 12]
The frequency of hepatic-related adverse events and hepatic-related treatment discontinuations will be tabulated from case report forms and will be based on unique subjects reporting such events.
6. The mean change from baseline in the MSQ role function [Time Frame: baseline to end of Week 12]
The mean change from baseline in the MSQ role function—restrictive domain score at Week 12 of the double-blind treatment phase.
7. The mean change from baseline in the MIDAS total score. [Time Frame: baseline to end of Week 12]
The mean change from baseline in the MIDAS total score at Week 12 of the double-blind treatment phase.

Further details concerning the clinical study including eligibility criteria, contacts and locations and more information can be found at www.clinicaltrials.gov for ClinicalTrials.gov Identifier: NCT03732638.

### Example 3

Results from the study described in Example 2 are shown in Tables 1-3 set forth below.

TABLE 1
Demographics and Baseline Characteristics
Treated Subjects.

| | | |
|---|---|---|
| Rimegepant | Placebo | Overall |
| (N = 370) | (N = 371) | (N = 741) |

Age at informed consent (years)

| n | 370 | | 371 | | 741 | |
|---|---|---|---|---|---|---|
| Mean (SD) | 41.3 | (13.01) | 41.1 | (13.13) | 41.2 | (13.06) |

| | | | |
|---|---|---|---|
| Median | 40.5 | 40.0 | 40.0 |
| Min, Max | 18.0, 74.0 | 18.0, 75.0 | 18.0, 75.0 |

Age at informed consent (years) category: n (%)

| | | | | | | |
|---|---|---|---|---|---|---|
| <40 | 177 | (47.8) | 180 | (48.5) | 357 | (48.2) |
| >=40 | 193 | (52.2) | 191 | (51.5) | 384 | (51.8) |
| <65 | 355 | (95.9) | 345 | (93.0) | 700 | (94.5) |
| >=65 | 15 | (4.1) | 26 | (7.0) | 41 | (5.5) |

Sex: n (%)

| | | | | | | |
|---|---|---|---|---|---|---|
| Female | 300 | (81.1) | 313 | (84.4) | 613 | (82.7) |
| Male | 70 | (18.9) | 58 | (15.6) | 128 | (17.3) |

Experiences menstrual periods (if female) *: n (%)

| | | | | | | |
|---|---|---|---|---|---|---|
| Yes | 191 | (63.7) | 204 | (65.2) | 395 | (64.4) |
| No | 109 | (36.3) | 109 | (34.8) | 218 | (35.6) |

Childbearing potential (if female) *: n (%)

| | | | | | | |
|---|---|---|---|---|---|---|
| Yes | 185 | (61.7) | 189 | (60.4) | 374 | (61.0) |
| No | 115 | (38.3) | 124 | (39.6) | 239 | (39.0) |

Race: n (%)

| | | | | | | |
|---|---|---|---|---|---|---|
| White | 295 | (79.7) | 309 | (83.3) | 604 | (81.5) |
| Black or African | 62 | (16.8) | 49 | (13.2) | 111 | (15.0) |
| American | | | | | | |
| Other including Asian | 13 | (3.5) | 13 | (3.5) | 26 | (3.5) |
| American Indian or | 6 | (1.6) | 1 | (0.3) | 7 | (0.9) |
| Alaska Native | | | | | | |
| Asian | 1 | (0.3) | 7 | (1.9) | 8 | (1.1) |
| Multiple | 6 | (1.6) | 2 | (0.5) | 8 | (1.1) |
| Native Hawaiian or | 0 | (0.0) | 3 | (0.8) | 3 | (0.4) |
| Other Pacific Islander | | | | | | |

Ethnicity: n (%)

| | | | | | | |
|---|---|---|---|---|---|---|
| Hispanic or Latino | 105 | (28.4) | 98 | (26.4) | 203 | (27.4) |
| Not Hispanic or Latino | 265 | (71.6) | 273 | (73.6) | 538 | (72.6) |

Country: n (%)

| | | | | | | |
|---|---|---|---|---|---|---|
| USA | 370 | (100.0) | 371 | (100.0) | 741 | (100.0) |

Screened for previous BHV3000 study: n (%)

| | | | | | | |
|---|---|---|---|---|---|---|
| Any previous study | 6 | (1.6) | 13 | (3.5) | 19 | (2.6) |
| BHV3000-201 | 1 | (0.3) | 1 | (0.3) | 2 | (0.3) |
| BHV3000-301 | 1 | (0.3) | 2 | (0.5) | 3 | (0.4) |
| BHV3000-303 | 4 | (1.1) | 10 | (2.7) | 14 | (1.9) |

Height (cm)

| n | 370 | | 371 | | 741 | |
|---|---|---|---|---|---|---|
| Mean (SD) | 165.9 | (8.72) | 165.9 | (8.48) | 165.9 | (8.59) |

| | | | |
|---|---|---|---|
| Median | 165.1 | 165.0 | 165.1 |
| Min, Max | 144.8, 193.0 | 149.3, 195.6 | 144.8, 195.6 |

Weight (kg)

| n | 370 | | 371 | | 741 | |
|---|---|---|---|---|---|---|
| Mean (SD) | 73.5 | (13.30) | 72.3 | (12.96) | 72.9 | (13.14) |

| | | | |
|---|---|---|---|
| Median | 73.0 | 72.1 | 72.6 |
| Min, Max | 42.4, 121.0 | 44.9, 107.0 | 42.4, 121.0 |

BMI (kg/m\hat{ }2)

| n | 370 | | 371 | | 741 | |
|---|---|---|---|---|---|---|
| Mean (SD) | 26.6 | (3.74) | 26.1 | (3.82) | 25.3 | (3.78) |

| | | | |
|---|---|---|---|
| Median | 26.8 | 26.5 | 26.8 |
| Min, Max | 18.2, 34.0 | 16.8, 32.9 | 16.8, 34.0 |

BMI (kg/m\hat{ }2) category: n (%)

| | | | | | | |
|---|---|---|---|---|---|---|
| <25 | 132 | (35.7) | 141 | (38.0) | 273 | (36.8) |
| >=25 to <30 | 161 | (43.5) | 170 | (45.8) | 331 | (44.7) |
| >=30 | 77 | (20.8) | 60 | (16.2) | 137 | (18.5) |

Prophylactic migraine medication use at randomization
(IWRS randomization stratum): n (%)

| | | | | | | |
|---|---|---|---|---|---|---|
| Yes | 81 | (21.9) | 83 | (22.4) | 164 | (22.1) |
| No | 289 | (78.1) | 288 | (77.6) | 577 | (77.9) |

* Percentages are based on the number of female subjects.

TABLE 2
Overall Summary of Primary and Secondary on Double-Blind Treatment
Evaluable mITT Subjects.

| | | Rimegapant | Placebo | Difference |
|---|---|---|---|---|
| Endpoint | Statistic | (N = 348) | (N = 347) | (Rimegepant − Placebo) |
| Primary Endpoint: Change | n | 348 | 347 | |
| From OP in Mean Number of | Least-squares mean | −4.5 | −3.7 | −0.8 |
| Total Migraine Days per Month | 95% CI | (−4.95, −3.97) | (−4.17, −3.20) | (−1.42, −0.14) |
| in Last Month * | P-value | | | 0.0176 @ |

Secondary Endpoints

| | | | | |
|---|---|---|---|---|
| >=50% Reduction From OP in | Response Rate (n/N) | 167/348 | 143/347 | |
| Mean Number of Moderate or | Stratified Risk # | 48.0 | 41.2 | 5.7 |
| Severe Migraine Days per | 95% CI | (42.7, 53.2) | (36.0, 46.4) | (−0.6, 14.1) |
| Month in Last Month | P-value | | | 0.0734 |
| Change From OP in Mean | n | 348 | 347 | |
| Number of Total Migraine Days | Least-squares mean | −3.5 | −2.8 | −0.7 |
| per Month Overall * | 95% CI | (−3.94, −3.12) | (−3.24, −2.42) | (−1.23, −0.17) |
| | P-value | | | 0.0094 |
| Rescue Medication Days per | n | 348 | 347 | |
| Month In Last Month * | Least-squares mean | 3.7 | 4.0 | −0.2 |
| | 95% CI | (3.29, 4.15) | (3.53, 4.39) | (−0.80, 0.31) |
| | P-value | | | 0.3868 |
| Change From OP in Mean | n | 348 | 347 | |
| Number of Total Migraine Days | Least-squares mean | −2.6 | −1.7 | −0.9 |
| per Month in First Month * | 95% CI | (−3.04, −2.17) | (−2.11, −1.25} | (−1.49, −0.36) |
| | P-value | | | 0.0013 |
| MSQoL Restrictive Role | n | 269 | 266 | |
| Function Domain Score Change | Least-squares mean | 18.0 | 14.6 | 3.5 |
| From Baseline at Week 12 ** | 95% CI | (15.54, 20.56) | (12.07, 17.10) | (0.23, 6.70) |
| | P-value | | | 0.0358 |
| MIDAS Total Score Change | n | 269 | 266 | |
| From Baseline at Week 12 ** | Least-squares mean | −11.8 | −11.7 | −0.1 |
| | 95% CI | (−15.41, −8.21) | (−15.29, −8.10) | (−4.74, 4.51) |
| | P-value | | | 0.9616 |

Evaluable subjects are those with >=14 days of eDiary efficacy data (not necessarily consecutive) in both the Observational Period (OP) and >=1 month (4-week interval) in the Double-Blind Treatment Phase.
* Generalized linear mixed effects model with treatment group, prophylactic migraine medication use at randomization, month, and month-by-treatment group interaction as fixed effects and subject as random effect
** Generalized linear model with treatment group and prophylactic migraine medication use at randomization as fixed effects and baseline score as covariate
# Stratified by prophylactic migraine medication use at randomization using Cochran-Mantel-Haenszel weighting
@ Significant in hierarchical testing

TABLE 3
Adverse Events on Double-Blind Treatment by Severity,
System Organ Class and Preferred Term
Treated Subjects.

Severity

| System Organ Class: n (%) | Rimegepant | | Placebo | |
|---|---|---|---|---|
| Preferred Term: n (%) | (N = 370) | | (N = 371) | |
| Total | | | | |
| Any AE | 133 | (35.9) | 133 | (35.8) |
| Infections and infestations | 51 | (13.8) | 55 | (14.8) |
| Nasopharyngitis | 13 | (3.5) | 9 | (2.4) |
| Urinary tract infection | 9 | (2.4) | 8 | (2.2) |
| Upper respiratory tract infection | 8 | (2.2) | 10 | (2.7) |
| Ear infection | 4 | (1.1) | 2 | (0.5) |
| Sinusitis | 4 | (1.1) | 11 | (3.0) |
| Fungal infection | 3 | (0.8) | 0 | |
| Influenza | 3 | (0.8) | 2 | (0.5) |
| Bacterial vaginosis | 2 | (0.5) | 2 | (0.5) |
| Bronchitis | 2 | (0.5) | 4 | (1.1) |
| Gastroenteritis | 2 | (0.5) | 1 | (0.3) |
| Hordepium | 2 | (0.5) | 0 | |
| Tonsillitis | 2 | (0.5) | 2 | (0.5) |
| Cervicitis | 1 | (0.3) | 0 | |
| Gastroenteritis viral | 1 | (0.3) | 1 | (0.3) |
| Herpes zoster | 1 | (0.3) | 1 | (0.3) |
| Lower respiratory tract infection | 1 | (0.3) | 0 | |
| Onychomycosis | 1 | (0.3) | 0 | |
| Pharyngitis | 1 | (0.3) | 0 | |
| Rhinitis | 1 | (0.3) | 0 | |
| Viral infection | 1 | (0.3) | 0 | |
| Viral upper respiratory tract infection | 1 | (0.3) | 0 | |
| Vulvovaginal mycotic infection | 1 | (0.3) | 0 | |
| Wound infection | 1 | (0.3) | 0 | |
| Acute sinusitis | 0 | | 1 | (0.3) |
| Appendicitis | 0 | | 1 | (0.3) |
| Enterobiasis | 0 | | 1 | (0.3) |
| Folliculitis | 0 | | 1 | (0.3) |
| Oral candidiasis | 0 | | 1 | (0.3) |
| Orchitis | 0 | | 1 | (0.3) |
| Otitis media | 0 | | 1 | (0.3) |
| Pelvic inflammatory disease | 0 | | 1 | (0.3) |
| Pertussis | 0 | | 1 | (0.3) |
| Pharyngitis streptococcal | 0 | | 2 | (0.5) |
| Pneumonia | 0 | | 1 | (0.3) |
| Pyelonephritis | 0 | | 1 | (0.3) |
| Sepsis | 0 | | 1 | (0.3) |
| Tooth infection | 0 | | 1 | (0.3) |
| Mild | | | | |
| Any AE | 92 | (24.9) | 90 | (24.3) |
| Infections and infestations | 31 | (8.4) | 31 | (8.4) |
| Nasopharyngitis | 10 | (2.7) | 7 | (1.9) |
| Urinary tract infection | 5 | (1.4) | 5 | (1.6) |
| Bacterial vaginosis | 2 | (0.5) | 0 | |
| Fungal infection | 2 | (0.5) | 0 | |
| Influenza | 2 | (0.5) | 2 | (0.5) |
| Sinusitis | 2 | (0.5) | 5 | (1.3) |
| Upper respiratory tract infection | 2 | (0.5) | 5 | (1.3) |
| Bronchitis | 1 | (0.3) | 2 | (0.5) |
| Cervicitis | 1 | (0.3) | 0 | |
| Ear infection | 1 | (0.3) | 0 | |
| Gastroenteritis viral | 1 | (0.3) | 1 | (0.3) |
| Herpes zoster | 1 | (0.3) | 0 | |
| Hordeolum | 1 | (0.3) | 0 | |
| Pharyngitis | 1 | (0.3) | 0 | |
| Rhinitis | 1 | (0.3) | 0 | |
| Tonsillitis | 1 | (0.3) | 0 | |
| Viral infection | 1 | (0.3) | 0 | |
| Viral upper respiratory tract infection | 1 | (0.3) | 0 | |
| Enterobiasis | 0 | | 1 | (0.3) |
| Folliculitis | 0 | | 1 | (0.3) |
| Oral candidiasis | 0 | | 1 | (0.3) |
| Orchitis | 0 | | 1 | (0.3) |
| Otitis media | 0 | | 1 | (0.3) |
| Moderate | | | | |
| Any AE | 64 | (17.3) | 62 | (16.7) |
| Infections and infestations | 23 | (6.2) | 28 | (7.5) |
| Upper respiratory tract infection | 6 | (1.6) | 5 | (1.3) |
| Urinary tract infection | 4 | (1.1) | 2 | (0.5) |
| Ear infection | 3 | (0.8) | 2 | (0.5) |
| Nasopharyngitis | 3 | (0.8) | 2 | (0.5) |
| Sinusitis | 2 | (0.5) | 6 | (1.6) |
| Bronchitis | 1 | (0.3) | 2 | (0.5) |
| Fungal infection | 1 | (0.3) | 0 | |
| Gastroenteritis | 1 | (0.3) | 0 | |
| Hordeolum | 1 | (0.3) | 0 | |
| Influenza | 1 | (0.3) | 0 | |
| Lower respiratory tract infection | 1 | (0.3) | 0 | |
| Onychomycosis | 1 | (0.3) | 0 | |
| Tonsillitis | 1 | (0.3) | 2 | (0.5) |
| Vulvovaginal mycotic infection | 1 | (0.3) | 0 | |
| Wound infection | 1 | (0.3) | 0 | |
| Acute sinusitis | 0 | | 1 | (0.3) |
| Appendicitis | 0 | | 1 | (0.3) |
| Bacterial vaginosis | 0 | | 2 | (0.5) |
| Herpes zoster | 0 | | 1 | (0.3) |
| Pharyngitis streptococcal | 0 | | 2 | (0.5) |
| Sepsis | 0 | | 1 | (0.3) |
| Moderate or Severe | | | | |
| Any AE | 68 | (18.4) | 64 | (17.3) |
| Infections and Infestations | 23 | (6.2) | 29 | (7.8) |
| Upper respiratory tract infection | 6 | (1.6) | 5 | (1.3) |
| Urinary tract infection | 4 | (1.1) | 2 | (0.5) |
| Ear infection | 3 | (0.8) | 2 | (0.5) |
| Nasopharyngitis | 3 | (0.8) | 2 | (0.5) |
| Gastroenteritis | 2 | (0.5) | 1 | (0.3) |
| Sinusitis | 2 | (0.5) | 6 | (1.6) |
| Bronchitis | 1 | (0.3) | 2 | (0.5) |
| Fungal infection | 1 | (0.3) | 0 | |
| Hordeolum | 1 | (0.3) | 0 | |
| Influenza | 1 | (0.3) | 0 | |
| Lower respiratory tract infection | 1 | (0.3) | 0 | |
| Onychomycosis | 1 | (0.3) | 0 | |
| Tonsillitis | 1 | (0.3) | 2 | (0.5) |

| | | | |
|---|---|---|---|
| Volvovaginal mycotic infection | 1 | (0.3) | 0 |
| Wound infection | 1 | (0.3) | 0 |

| | | | |
|---|---|---|---|
| Acute sinusitis | 0 | 1 | (0.3) |
| Appendicitis | 0 | 1 | (0.3) |
| Bacterial vaginosis | 0 | 2 | (0.5) |
| Herpes zoster | 0 | 1 | (0.3) |
| Pharyngitis streptococcal | 0 | 2 | (0.5) |
| Pneumonia | 0 | 1 | (0.3) |
| Pyelonephritis | 0 | 1 | (0.3) |
| Sepsis | 0 | 1 | (0.3) |

Medical Dictionary for Regulatory Activities (MedDRA) Version 21.1
Adverse events (AEs) are listed in descending order of rimegepant frequency within system organ class and preferred term.

The Study described in Example 2 demonstrated positive topline results in the randomized, placebo-controlled pivotal clinical trial (NCT03732638) evaluating the efficacy and safety of oral rimegepant 75 mg for the preventive treatment of migraine in both episodic and chronic migraine patients. The study met the primary endpoint, demonstrating a statistically significant reduction from baseline in monthly migraine days in patients treated with rimegepant compared with placebo. Those receiving rimegepant 75 mg every other day experienced a statistically significant 4.5 day reduction from baseline in monthly migraine days, compared to a 3.7 day reduction in the placebo group (p=0.0176). A total of 22% of the study participants were taking a concurrent preventive treatment including topiramate and amitriptyline. In those study participants not taking concomitant preventive treatment, the change from all baseline migraine days was a 4.9 day reduction from baseline in monthly migraine days in the rimegepant group (n=273), compared to a 3.7 day reduction in the placebo group (n=269). Importantly, 48% of the rimegepant group had at least a 50% reduction from baseline in the mean number of moderate to severe migraine days per month compared to 41% in the placebo group. The reduction in mean number of migraine days in accordance with the present invention may, for example, be at least 20%, 30%, 40% or 50% as compared to placebo. For example, (4.9−3.7)/3.7*100=32%.

This pivotal study enrolled patients with both episodic and chronic migraine. The study evaluated the efficacy and safety of rimegepant 75 mg (n=370) dosed every other day for the preventive treatment of migraine in patients versus placebo (n=371) who had migraine for at least one year and 4 to 18 moderate to severe migraine attacks per month over three months prior to enrollment. During the one-month observation period, patients experienced on average 10.7 migraine days per month, with 7.4 migraine days of moderate to severe migraine during the same period.

These data demonstrate rimegepant's broad range of clinical activity to potentially provide a new oral preventive treatment option for people with migraine. The magnitude of effect in the rimegepant treated arm of a 4.5 day reduction in this study was consistent with other approved preventive drugs for migraine and also with our own large open-label Phase 2 trial of rimegepant which previously showed up to a 5.3 day reduction in migraine days per month in patients with moderate to severe intensity migraine. The goal of our strategy is to deliver one medication to address patients' needs for treating their migraines. The efficacy and safety results suggest that rimegepant may be an ideal first-line, oral therapy for both preventive and acute treatment of migraine.

The safety profile seen in the 370 patients who received rimegepant 75 mg every other day was consistent with prior clinical trial experience. There were no cases of ALT or AST>3×ULN and bilirubin>2×ULN. An independent liver monitoring panel did not determine any ALT/AST elevations to be in the categories of probably or definitely related to study drug.

The bioequivalence of rimegepant 75 mg in a tablet form such as described in Example 1 herein has been demonstrated clinically, such as described in Example 5 of WO 2019/191008 A1, published Oct. 3, 2019. The safety and efficacy of rimegepant 75 mg in an orally disintegrating tablet (ODT) form has been demonstrated clinically, such as described in the clinical trial “BHV3000-303: Phase 3: Double-Blind, Randomized, Placebo-Controlled, Safety and Efficacy Trial of BHV-3000 (Rimegepant) Orally Disintegrating Tablet (ODT) for the Acute Treatment of Migraine” (ClinicalTrials.gov Identifier: NCT03461757).

The safety and efficacy of rimegepant 75 mg in an orally disintegrating tablet (ODT) form has been further described in the article by Robert Croop, Richard B. Lipton, David Kudrow, David A. Stock, Lisa Kamen, Charles M. Conway, Elyse G. Stock, Vladimir Coric, Peter J. Goadsby “Oral rimegepant for preventive treatment of migraine: a phase 2/3, randomised, double-blind, placebo-controlled trial” published in Lancet 2021, 397 (10268), 51-60. doi: 10.1016/S0140-6736 (20) 32544-7, which is incorporated herein in its entirety by reference.

Throughout this application, various publications are referenced by author name and date, or by patent number or patent publication number. The disclosures of these publications are hereby incorporated in their entireties by reference into this application in order to more fully describe the state of the art as known to those skilled therein as of the date of the invention described and claimed herein. However, the citation of a reference herein should not be construed as an acknowledgement that such reference is prior art to the present invention.

Those skilled in the art will recognize, or be able to ascertain using no more than routine experimentation, numerous equivalents to the specific procedures described herein. Such equivalents are considered to be within the scope of this invention and are covered by the following claims. For example, pharmaceutically acceptable salts other than those specifically disclosed in the description and Examples herein can be employed. Furthermore, it is intended that specific items within lists of items, or subset groups of items within larger groups of items, can be combined with other specific items, subset groups of items or larger groups of items whether or not there is a specific disclosure herein identifying such a combination.

What is claimed is:

1. A method for the preventative treatment migraine in a patient in need thereof, comprising administering to the patient a pharmaceutical composition comprising a pharmaceutically acceptable carrier and a therapeutically effective amount of rimegepant, or a pharmaceutically acceptable salt thereof.

2. The method of claim 1, wherein administering the pharmaceutical composition provides a reduction in the mean number of migraine days per month for said patient.

3. The method of claim 1, wherein the pharmaceutical composition is administered at least once every other day.

4. The method of claim 2, wherein the reduction in the mean number of migraine days per month for said patient at least 20%.

5. The method of claim 2, wherein the reduction in the mean number of migraine days per month for said patient at least 30%.

6. The method of claim 2, wherein the reduction in the mean number of migraine days per month for said patient at least 40%.

7. The method of claim 2, wherein the reduction in the mean number of migraine days per month for said patient at least 50%.

8. The method of claim 3, wherein the pharmaceutical composition is administered once every other day.

9. The method of claim 3, wherein the pharmaceutical composition is administered not more than once per day.

10. The method of claim 1, wherein the patient is also administered a medication for the treatment of acute migraine.

11. The method of claim 10, wherein the medication is a CGRP inhibitor selected from a CGRP antibody, a CGRP receptor antibody, an antigen-binding fragment from a CGRP antibody or a CGRP receptor antibody, a CGRP infusion inhibitory protein, a CGRP bio-neutralizing agent, a CGRP receptor antagonist, a small molecule CGRP inhibitor, or a polypeptide CGRP inhibitor.

12. The method of claim 11, wherein the CGRP antibody is selected from galcanezumab-gnlm, fremanezumab-vfrm, eptinezumab-jjmr, and erenumab-aooe.

13. The method of claim 11, wherein the CGRP receptor antagonist is selected from olcegepant, telcagepant, ubrogepant, atogepant, rimegepant, and zavegepant.

14. The method of claim 1, wherein the rimegepant is in the form of a hemisulfate sesquihydrate salt.

15. The method of claim 1, wherein the pharmaceutical composition is in the form of a tablet.

16. The method of claim 15, wherein the pharmaceutical composition comprises from about 50-60 weight % rimegepant hemisulfate sesquihydrate, about 30-35 weight % microcrystalline cellulose, about 2-7 weight % hydroxypropyl cellulose, about 3-7 weight % croscarmellose sodium, and about 0.1-1.0 weight % magnesium stearate.

17. The method of claim 16, wherein the pharmaceutical composition comprises about 57.1 weight % rimegepant hemisulfate sesquihydrate, about 33.4 weight % microcrystalline cellulose, about 4.0 weight % hydroxypropyl cellulose, about 5.0 weight % croscarmellose sodium, and about 0.5 weight % magnesium stearate.

18. The method of claim 1, wherein the pharmaceutical composition is in the form of an oral solid molded fast-dispersing dosage form.

19. The method of claim 18, wherein the pharmaceutical composition comprises from about 70-80 weight % rimegepant hemisulfate sesquihydrate, about 10-20 weight % fish gelatin, about 10-20 weight % of a filler, and 0.1-5.0 weight % of a flavorant.

20. The method of claim 19, wherein the filler is mannitol.

21. A method of treating sinusitis in a patient in need thereof, comprising administering to the patient a pharmaceutical composition comprising a pharmaceutically acceptable carrier and a therapeutically effective amount of rimegepant, or a pharmaceutically acceptable salt thereof, in order to relieve a symptom associated with the sinusitis.

22. The method of claim 21, wherein the treatment is effective to provide pain relief to the patient.

23. The method of claim 21, wherein the treatment is effective to reduce an infection in the patient.

24. The method of claim 21, wherein the treatment is effective to reduce inflammation in the patient.
